(19) United States
(12) Patent Application Publication
(10) Pub. No.: US 2019/0060212 A1
(43) Pub. Date: Feb. 28, 2019

(54) Cosmetic Cream with Anti-Oxidant Activity

(71) Applicants: Roman Aleksandrovich, Brooklyn, NY (US); Oleg Ponomarev, Englishtown, NJ (US)

(72) Inventors: Roman Aleksandrovich, Brooklyn, NY (US); Oleg Ponomarev, Englishtown, NJ (US)

(21) Appl. No.: 16/104,437

(22) Filed: Aug. 17, 2018

Related U.S. Application Data
(60) Provisional application No. 62/549,145, filed on Aug. 23, 2017.

(51) Int. Cl. A61K 8/92; A61K 8/99; A61K 8/49; A61K 8/73; A61K 8/34; A61K 8/60; A61Q 17/04; A61Q 19/08; A61Q 19/00

(52) U.S. Cl. CPC A61K 8/922; A61K 8/99; A61K 8/498; A61K 8/738; A61K 8/342; A61K 2800/522; A61K 8/345; A61Q 17/04; A61Q 19/08; A61Q 19/008; A61K 2800/592; A61K 8/602

(57) ABSTRACT

The proposed cosmetic cream formula contains predetermined quantities of the following ingredients: Grapeseed oil, Spirulina extract, Taxifolin/Dihydroquercetin, Beta-Cyclodextrine, Cetearyl Alcohol, Cetearyl Glucoside, 1,2 Propylene Glycol, Phenylethyl Alcohol, Benzyl Alcohol, Sorbin Acids and Distilled water. The presence of Spirulina extract and Taxifolin in water soluble form in the oil-water base formula of the proposed cosmetic cream provides for a synergetic effect that greatly enhances Spirulina's skin protection properties. There is also disclosed a preferable content of ingredients for preparation of the Spirulina extract.

### CROSS REFERENCES TO RELATED APPLICATIONS

The present patent application claims the benefit of a U.S. provisional patent application Ser. No. 62/549,145 filed on Aug. 23, 2017, the disclosure of which is incorporated herein in its entirety by reference.

### FIELD OF THE INVENTION

The invention relates to cosmetic chemistry formulations, especially to new effective skin creams and lotions formulations and their properties. In particular, the invention relates to creating new skin care cosmetic formulas.

### BACKGROUND OF THE INVENTION

Currently, cosmetic cream and lotion formulations commonly use natural bioflavonoids including Taxifolin with proved antioxidant properties. It is already clear that adding one, or a combination of bioflavonoids greatly improves the quality of cosmetic products on their base by adding new and enhancing properties that were previously known for protecting skin from UV light, aging, wrinkles, acne and extra oil production. Examples of such known products are described in the following articles:

Anti wrinkle skin care composition containing antioxidant peptides.

PATENT PAK. By Jannsen, Frank. From Ger. Offen. (2010), DE102008061044 A1 Jun. 17, 2010/Language: German, Database: CAPSUL;

Anti-oxidative composition against skin aging ciontaining triphenyl phosphonium coupled antioxidants. PATENTPAK. By Janssen, Frank. From Ger. Offen. (2010), DE 102008061340 A1 Sep. 23, 2010/Language: German, Dtabase: CAPSUL;

Cosmetic and dermatilogical combinations of Carnitine or acylcarnitines with antioxidants.

PATENTPAK. By Staeb, Franz, Schoenrock, Uwe; Schreiner, Volker; Max, Heiner, Untied, Sven From Ger. Offen. (1999) DE 19806890 A1 Aug. 26, 1999/Language: German, Dtabase: CAPSUL;

Taxifolin (5,7,31,41-tetrahydroxyflavanol,dihydroquercetin) belongs to amemberoftheflavonoids family. Taxifolin was found in the leaves of Chamaecyparisobtuse (Cupressaceae) and also is commonly extracted from Pseudotsuga taxifolia (Lamb.) Britt., Larix gmelinii (Rupr.) Kuzen, and Larix sibirica Ledeb. (Pinaceae) [1-3] (see [References] herein below).

It was even obtained from fruits, vegetables, beverages, and so on. It elicits a wide range of pharmacological effects of anti-oxidation and anti-radiation [4,5].

Furthermore, it also has anti-inflammation activity, anti-viral activity, anti-tumor activity, and protective postmenopausal osteoporosis activity [6-9].

Due to its pharmacological diversity, its bioavailability and biological properties have raised a great interest in the following studies ([References]):
1. Fukui, Y.; Nakadome, K.; Ariyoshi, H. Studies on the monomer flavonoides of the plants of coniferae. II. Isolation of a new taxifolin glucoside from the leaves of Chamaecyparis obtusa Endlicher. Yakugaku Zasshi. 1966, 86, 184-187. [PubMed]
2. Grimm, T.; Skrabala, R.; Chovanova, Z.; Muchova, J.; Sumegova, K.; Liptakova, A.; Durackova, Z.; Hogger, P. Single and multiple dose pharmacokinetics of maritime pine bark extract (pycnogenol) after oral administration to healthy volunteers. BMC Clin. Pharmacol. 2006, 6. [CrossRef] [PubMed]
3. Weidmann, A. E. Dihydroquercetin: Morethanjustanimpurity. Eur. J. Pharmacol. 2012, 684, 19-26. [CrossRef] [PubMed].
4. Sun, X.; Chen, R. C.; Yang, Z. H.; Sun, G. B.; Wang, M.; Ma, X. J.; Yang, L. J.; Sun, X. B. Taxifolinpreventsdiabetic cardiomyopathy in vivo and in vitro by inhibition of oxidative stress and cell apoptosis. FoodChem. Toxicol. 2013, 63, 221-232. [CrossRef] [PubMed]
5. Tiukavkina, N. A.; Rulenko, I. A.; Kolesnik, I. U. A. Dihydroquercetin-anewantioxidantandbiologicallyactive food sdditive. J. Vopr. Pitan. 1997, 6, 12-15.
6. Oi, N.; Chen, H. Y.; Kim, M. O.; Lubet, R. A.; Bode, A. M.; Dong, Z. G. Taxifolin suppresses UV-induced skin carcinogenesis by targeting EGFR and PI3K. Cancer Prev. Res. 2012, 5, 1103-1114. [CrossRef] [PubMed].
7. Satue, M.; Arriero, M. M.; Monjo, M.; Ramis, J. M. Quercitrin and taxifolin stimulate osteoblast differentiation inMC3T3-ElcellsandinhibitosteoclastogenesisinRAW264. 7 cells. Biochem. Pharmacol. 2013, 86, 1476-1486. [CrossRef] [PubMed].
8. Tamara, V. A.; Antonina, F. K.; Ludmila, N. K.; Maria, K. L.; Vera, V. S.; Yuri, N. K.

Effects of taxifolin on the activity of angiotensin-converting enzyme and reactive oxygen and nitrogen species in the aorta of aging rats and rats treated with the nitric oxide synthase. Age 2013, 35, 2089-2097.
9. Verma, S.; Singh, A.; Mishra, A. Dual inhibition of chaperoning process by taxifolin: Molecular dynamics imulation study. J. Mol. Graph. Model. 2012, 37, 27-38. [CrossRef] [PubMed].

Currently, cosmetic cream and lotion formulations commonly use natural bioflavonoids including Taxifolin with proved antioxidant properties. It is already clear that adding one, or a combination of bioflavonoids greatly improves the quality of cosmetic products on their base by adding new and enhancing properties that were previously known for protecting skin from UV light, aging, wrinkles, acne and extra oil production.

All currently existing formulations with Taxifolin and other flavonoids are based on standard formulas using cosmetic fatty acids, vitamins, and up to 30-60 other natural and synthetic ingredients for advanced cream and lotions.

All of those ingredients have been studied and have been strongly proven for use in cosmetic formulations, but very often lose their properties in a multi (50-60) ingredients formula.

The specific chemical and physical interactions, along with synergetic effects of many ingredients in complicated formulas have not been studied, and are currently not strongly proven and defined. It is becoming clear that very often only certain ingredients of multicomponent cosmetic formulas provide their claimed effects, and all others exist just for commercial purpose.

Using synthetic preservatives like BHA, BHT and others for most cosmetic formulas is also a well-known drawback of all cosmetic formulations containing these preservatives. The contemporary trend is to substitute those ingredients for natural preservative to prevent these drawbacks.

Bioavailability of current cosmetic and pharmaceutical ingredients is also becoming a main factor of efficiency formulated products and taking greater attention of scientists working in this field. Development of new cosmetic formulas with synergetic effect between ingredients and high bioavailability currently becomes very important for new effective skin care and pharmaceutical products.

### OBJECT AND SUMMARY OF THE INVENTION

Therefore, a primary object of the present invention is the obtaining of novel cosmetic skin care formula on the base of water-oil formulation with Spirulina extract, Grapeseed oil and bioavailable water soluble natural antioxidant (Taxifolin), which formula provides for a synergetic effect of all its ingredients and enhanced power of protecting human skin from UV light, aging, wrinkles, acne and extra oil production.

Specifically, structural formulas of Taxifolin and its analogs are illustrated in FIGS. 1-3 of the instant disclosure. They were used by the authors in a water soluble form as a complex formed with β-Cyclodextrin or amino acid like L-arginine, and used in the inventive water base oil cosmetic cream. The authors observed that the inventive formulation produced a homogenous structure with a synergetic effect of all ingredients of the cosmetic cream and enhanced its power of protecting skin from the above mentioned factors.

### DRAWINGS OF THE INVENTION

The present invention is illustrated in the attached drawings.

FIG. 1 shows a chemical structure of Taxifolin (Dihydroquercetin) and its analogues wherein:

R1═R3═R4═R6═R7═OH, R2═R5═H; R2═H, R1═R3═R4═R6═R7═OH, according to an embodiment of the invention.

FIG. 2 shows a chemical structure of Quercetin and its analogues wherein:

R1═R3═R4═R6═R7═OH, R2═R5═H; R2═H, R1═R3═R4═R6═R7═OH, according to another embodiment of the invention.

FIG. 3 shows a chemical structure wherein: R2═R5═H, R1═R3═R6═R7═OH, R4═O-diglucoside, according to another embodiment of the invention.

### DETAIL DESCRIPTION OF THE INVENTION

While the invention may be susceptible to embodiment in different forms, there are shown in the drawings, and will be described in detail herein, specific embodiments of the present invention, with the understanding that the present disclosure is to be considered an exemplification of the principles of the invention, and is not intended to limit the invention to that as illustrated and described herein.

The above stated object has been achieved in the present invention by using an oil-in-water technique formulation.

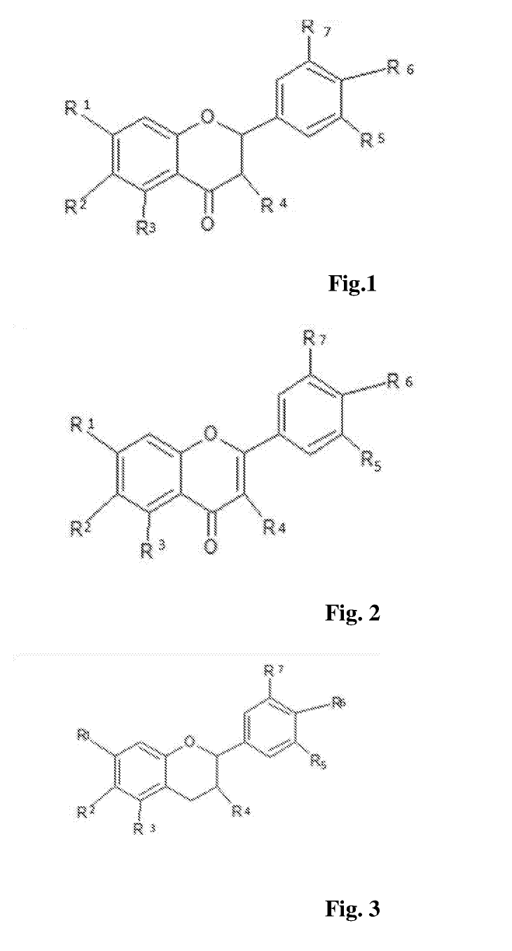

Except Taxifolin, the structures shown in FIGS. 1-3 can be used as an antioxidant in the inventive formula. Solubility of flavonoids in water is achieved with a formation inclusive complex with β-Cyclodextrin at a temperature of 40-45 C.

Spirulina platensis (SP), a blue-green algae (photosynthesizing cyanobacterium) having diverse biological activity is biopolymer cyanobacterium that is also used in the inventive cream formula.

Due to a high content of highly valuable proteins, indispensable amino acids, vitamins, beta-carotene and other pigments, mineral substances, indispensable fatty acids and polysaccharides, SP has been found suitable for use as bioactive additive. SP produces an immune stimulating effect by enhancing the resistance of mammals and fish to infections and has the capacity to influence hemopoiesis, stimulating the production of antibodies and cytokines.

Under the influence of SP macrophages, T and B cells are activated. SP sulfolipids have proved to be effective against HIV. Preparations obtained from SP biomass have also been found active against herpes virus, cytomegalovirus, influenza virus, etc. SP extracts are capable in inhibiting cancerogenesis.

SP preparations are regarded as functional products contributing to the preservation of the resident intestinal microflora, especially lactic acid bacilli and bifidobacteria, and to a decrease in the level of Candida albicans. The biological activity of SP with respect to microorganisms holds good promise in using these microalgae as components of culture media.

Although Spirulina contains a lot of useful ingredients that are usually used in cosmetic formulas in separate forms, Spirulina was never used in cosmetic formulas with water soluble bioflavonoids like antioxidants. However, the authors have established that flavonoid antioxidant present in a water-soluble form with enhanced bioavailability helps greatly increase Spirulina's skin protection properties.

Spirulina extract used for the inventive formulation of cream has the following dry weight content (nutritional value per 100 g/3.5 oz):

Carbohydrates 23.9 g; Sugars 3.1 g; Dietary fiber 3.6 g; Fat 7.72 g; Saturated fat 2.65 g; Monounsaturated fat 0.675 g; Polyunsaturated 2.08 g; Protein 57.47 g; Tryptophan 0.929 g; Threonine 2.97 g; Isoleucine 3.209 g; Leucine 4.947 g; Lysine 3.025 g; Methionine 1.149 g; Cystine 0.662 g; Phenylalanine 2.777 g; Tyrosine 2.584 g; Valine 3.512 g; Valin 3.512; Arginine 4.147 g; Histidine 1.085 g; Alanine 4.515 g; Aspartic acid 5.793 g; Glutamic acid 8.386 g; Glycine 3.099 g; Proline 2.382 g; Serine 2.998 g;

| Vitamins: | | |
|---|---|---|
| Vitamin A equiv | 29 | μg; |
| beta-carotene | 342 | μg; |
| Thiamine (B1) | 2.38 | mg; |
| Riboflavin (B2) | 3.67 | mg; |
| Niacin (B3) | 12.82 | mg; |
| Pantothenic acid (B5) | 3.48 | mg; |
| Vitamin B6 | 0.364 | mg; |
| Folate (B9) | 94 | μg; |
| Choline | 66 | mg; |
| Vitamin C | 10.1 | mg; |
| Vitamin E | 5 | mg; |
| Vitamin K | 25.5 | μg; |
| Minerals: | | |
| Calcium | 120 | mg; |
| Iron | 28.5 | mg; |
| Magnesium | 195 | mg; |
| Manganese | 1.9 | mg; |
| Phosphorus | 118 | mg; |
| Potassium | 1363 | mg; |
| Sodium | 1048 | mg; and |
| Zinc | 2 | mg. |

Energy value of 100 g of the formulation is 1.213 kJ (290 kcal)

The inventive formula also includes Grapeseed oil (Vitis Vinifera Oil) as a basic ingredient. Grapeseed oil is often used as a cosmetic ingredient for damaged or stressed tissues because it has regenerative qualities, specifically for collagen regeneration and capillary support. It is especially effective for repair of the skin around the eyes, and in the repair of stretch marks.

Grapeseed oil contains a high amount of linoleic acid, nourishing to skin anti-inflammatory, antiseptic and mildly astringent. Grapeseed oil also contains antioxidant polyphenol, which, in a mixture with Taxifolin, synergistically enhances their mutual antioxidant activity, which further contributes into the resultant synergistic effect of the inventive formula. Grapeseed oil is rich in linoleic acid, oleic acid, palmitic acid, stearic acid and palmit oleic acid. Additionally, Grapeseed oil contains vitamin E, C, D and Beta-carotene.

These qualities make Grapeseed oil especially valuable for antiaging skin care. However, since Grapeseed oil is a light oil, it is suitable for all skin types, including oily skin.

The inventive formula also includes usual cosmetic ingredients used for stability of cosmetic products: Cetearyl Alcohol, Cetearyl Gluco side 1.2, Propylene Glycol, Phenylethyl Alcohol, Benzyl Alcohol, and Sorbin Acids.

The TABLE below lists the preferable content of all ingredients of the cream formula in weight % for 1 kg of the inventive cosmetic cream:

| | | |
|---|---|---|
| 1 | Distilled water | 0.20-0.90 |
| 2 | Grape seeds oil (Vitis | 0.10-0.90 |
| | Vinifera Oil) | |
| 3 | Spirulina extract | 0.001-0.01 |
| 4 | Taxifolin/ | 0.001-0.10 |
| | Dihydroquercetin | |
| 5 | Beta-Cyclodextrine | 0.001-0.10 |
| 8 | Cetearyl Alcohol, Cetearyl | 0.01-1 |
| | Glucoside,1,2 Propylene | |
| | Glycol, Phenylethyl | |
| | Alcohol, Benzyl Alcohol, | |
| | Sorbin Acids | |

The claimed invention is:

1. A cosmetic cream, comprising (in weight % for 1 kg):
Grapeseed oil (Vitis Vinifera Oil): 0.10-0.90;
Spirulina extract: 0.001-0.01;
Taxifolin/Dihydroquercetin: 0.001-0.1;
Beta-Cyclodextrine: 0.001-0.1;
Cetearyl Alcohol, Cetearyl Glucoside, 1,2 Propylene Glycol, Phenylethyl Alcohol, Benzyl Alcohol, Sorbin Acids: variable from 0.001 to 1; and
Distilled water: 0.20-0.90.

2. The cosmetic cream in claim 1, wherein the Spirulina extract has the following dry content (nutritional value per 100 g/3.5 oz):
Carbohydrates 23.9 g; Sugars 3.1 g; Dietary fiber 3.6 g; Fat 7.72 g; Saturated fat 2.65 g; Monounsaturated fat 0.675 g; Polyunsaturated 2.08 g; Protein 57.47 g; Tryptophan 0.929 g; Threonine 2.97 g; Isoleucine 3.209 g; Leucine 4.947 g; Lysine 3.025 g; Methionine 1.149 g; Cystine 0.662 g; Phenylalanine 2.777 g; Tyrosine 2.584 g; Valine 3.512 g; Valin 3.512; Arginine 4.147 g; Histidine 1.085 g; Alanine 4.515 g; Aspartic acid 5.793 g; Glutamic acid 8.386 g; Glycine 3.099 g; Proline 2.382 g; Serine 2.998 g;

| Vitamins: | | |
|---|---|---|
| Vitamin A equiv | 29 | μg; |
| beta-carotene | 342 | μg; |
| Thiamine (B1) | 2.38 | mg; |
| Riboflavin (B2) | 3.67 | mg; |
| Niacin (B3) | 12.82 | mg; |
| Pantothenic acid (B5) | 3.48 | mg; |
| Vitamin B6 | 0.364 | mg; |
| Folate (B9) | 94 | μg; |
| Choline | 66 | mg; |
| Vitamin C | 10.1 | mg; |
| Vitamin E | 5 | mg; |
| Vitamin K | 25.5 | μg; |
| and Minerals: | | |
| Calcium | 120 | mg; |
| Iron | 28.5 | mg; |
| Magnesium | 195 | mg; |
| Manganese | 1.9 | mg; |
| Phosphorus | 118 | mg; |
| Potassium | 1363 | mg; |
| Sodium | 1048 | mg; and |
| Zinc | 2 | mg. |
